(19) United States
(12) Patent Application Publication
(10) Pub. No.: US 2024/0343757 A1
(43) Pub. Date: Oct. 17, 2024

(54) PROCESS FOR THE PREPARATION OF BUDESONIDE 21-PHOSPHATE

(71) Applicant: GENETIC S.P.A., Castel San Giorgio (IT)

(72) Inventors: Giuseppe CALIENDO, Marigliano (IT); Angela CORVINO, Caserta (IT); Ferdinando FIORINO, Benevento (IT); Francesco FRECENTESE, Aversa (IT); Elisa MAGLI, Portici (IT); Elisa PERISSUTTI, Venafro (IT); Antonio PETTI, Battipaglia (IT); Vincenzo SANTAGADA, Napoli (IT); Beatrice SEVERINO, Mugnano di Napoli (IT)

(21) Appl. No.: 18/579,645

(22) PCT Filed: Jul. 20, 2022
(86) PCT No.: PCT/EP2022/070381

§ 371 Date: Jan. 16, 2024

(30) Foreign Application Priority Data
Jul. 22, 2021 (IT) 102021000019454

(51) Int. Cl. C07J 71/00

(52) U.S. Cl. CPC C07J 71/0031

(57) ABSTRACT

The present invention relates to a new process for the preparation of budesonide 21-phosphate and its disodium salt.

### TECHNICAL FIELD

The present invention relates to a new process for the preparation of budesonide 21-phosphate and its disodium salt

### BACKGROUND OF THE INVENTION

Budesonide (Bud) (chemical name 1β,21-dihydroxy-16α, 17α-(butylidenebis(oxy)) pregna-1,4-diene-3,20-dione), is a glucocorticoid steroid for the treatment of asthma, chronic obstructive pulmonary disease (COPD), noninfectious rhinitis and Crohn disease, represented by Formula I.

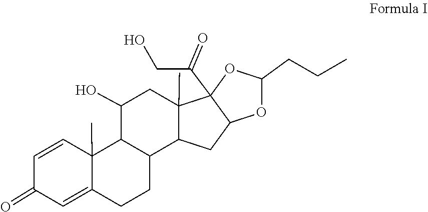 [SMILES: CCCC1OC2CC3C4CCC5=CC(=O)C=CC5(C)C4C(O)CC3(C)C2(C(=O)CO)O1]

Budesonide has a logP of 3.2 and results practically insoluble in water (28 μg/mL) [Journal of Chemical and Engineering Data (2010), vol. 55, no. 1, pp. 578-582] at physiological pH in the intestinal region. It belongs to inhaled corticosteroids (ICS), a class of compounds that represents, by far, the most effective therapeutic tool used in the treatment of asthma, able to suppress and activate many genes relevant to elicit inflammation in asthmatic airways, even in very low doses.

Budesonide is virtually insoluble in water while it results readily soluble in alcohols. For this reason, hydroalcoholic solutions are usually prepared dissolving an adequate amount of active substance in solubilizers such as water-soluble alcohols. However, the so prepared solutions have low stability because large amounts of budesonide are decomposed within a short time. Moreover, budesonide formulations have been prepared until now in the form of aqueous suspensions in which the solid phase tends in time to deposit onto the bottom of the container, thus requiring chemical additives or vigorous stirring. These are the reasons that make budesonide not suitable to be delivered by an electric nebulizer.

The 21-phosphate primary esters of several corticosteroids have been prepared and largely used as active ingredients for several pharmaceutical compositions. These molecules have valuable properties not possessed by the parent steroid; first, they are water soluble thus allowing the administration in aqueous solution.

Budesonide 21-phosphate (Formula II) has been used in some studies [Bioconjugate Chem. 2016, 27, 2081-2088; J. Am. Chem. Soc. 2016, 138, 1430-1445] where it is described as linker for targeted delivery of antibody-drug conjugates.

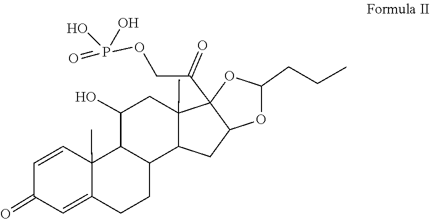 [SMILES: CCCC1OC2CC3C4CCC5=CC(=O)C=CC5(C)C4C(O)CC3(C)C2(C(=O)COP(=O)(O)O)O1]

Budesonide 21-phosphate disodium salt (Formula III) has been used in some studies for the preparation of liposomal glucocorticoids studied as antitumor agents [Journal of Steroid Biochemistry & Molecular Biology 111 (2008) 101-110; Journal of Controlled Release 127 (2008) 131-136].

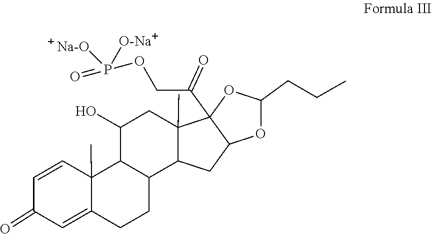

J. Am. Chem. Soc. 2016, 138, 1430-1445 473 describes the synthesis of Budesonide 21-phosphate starting from a stirred solution of budesonide in THF at −40° C. and reacting with diphosphoryl chloride. The reaction is quenched with water and treated with saturated sodium bicarbonate solution until pH˜8. The solution is subsequently made acidic using a 1 N HCl solution and extracted several times with ethyl acetate (3.55 g, 75%).

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

The terms “approximately” and “about” herein refer to the range of the experimental error, which may occur in a measurement.

The term “room temperature” herein refers to a temperature between 15° C. and 25° C.

The terms “comprising”, “having”, “including” and “containing” are to be construed open-ended terms (i.e. meaning “including, but not limited to”) and are to be considered as providing support also for terms as “consist essentially of”, “consisting essentially of”, “consist of” or “consisting of”.

The terms “consist essentially of”, “consisting essentially of” are to be construed as semi-closed terms, meaning that no other ingredients which materially affects the basic and novel characteristics of the invention are included (optional excipients may thus included).

The terms “consists of”, “consisting of” are to be construed as closed terms.

The term “unknown impurity” refers to any unknown impurity present in Budesonide 21-phosphate disodium salt.

The term “area %” herein refers to area under the curve in the HPLC chromatogram. References herein to percent (%) purity and impurity are based on area.

### SUMMARY OF THE INVENTION

The invention relates to a novel and efficient process that leads to Budesonide 21-phosphate and its disodium salt, which is convenient for the industrial scale and provides the desired products in good yields.

The process of the invention is described in Scheme 1.

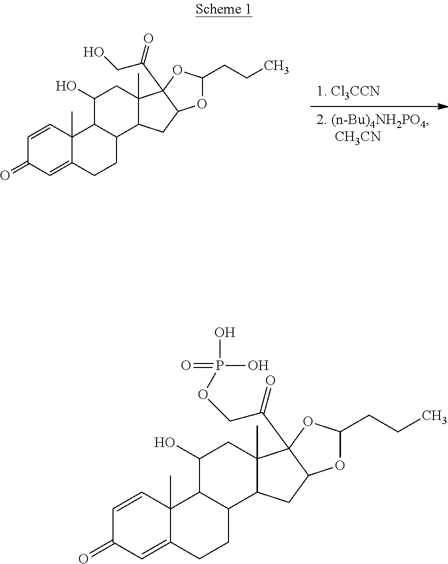 [SMILES: CCCC1OC2CC3C4CCC5=CC(=O)C=CC5(C)C4C(O)CC3(C)C2(C(=O)CO)O1 ; "1. Cl3CCN" ; "2. (n-Bu)4NH2PO4,     CH3CN" ; CCCC1OC2CC3C4CCC5=CC(=O)C=CC5(C)C4C(O)CC3(C)C2(C(=O)COP(=O)(O)O)O1]

Scheme 1 shows the one-pot process for the preparation of Budesonide 21-phosphate characterized by a phosphorylation with tetrabutylammonium dihydrogen phosphate and trichloroacetonitrile.

Preferably, the final product is isolated as disodium salt.

Conversely, the process described in the prior art involves the use of diphosphoryl chloride as a phosphorylating agent. The latter reacts with water to produce HCl and H₃PO₄. This makes it necessary to preserve both the reactive and the reaction mixture from contact with moisture. The known process requires more careful preparation of the reaction mixture and, therefore, more expensive chemical procedures.

Furthermore, the diphosphoryl chloride gives rise in the phosphorylation process to a strongly exothermic reaction which requires operating temperatures of −40° C. To guarantee this condition, expensive experimental methodologies are required both from an energy point of view and from the specialized personnel employed.

The process of the present invention is a notable improvement with respect to the prior art because the reaction takes place under very mild conditions and at room temperature. Therefore, it is more manageable, less expensive and safer. Furthermore, under such experimental conditions, no strong acids are produced.

### DESCRIPTION OF THE FIGURES

FIG. 1 shows 1H-NMR (500 MHZ; CD₃OD-d₄) spectrum of Budesonide 21-phosphate.

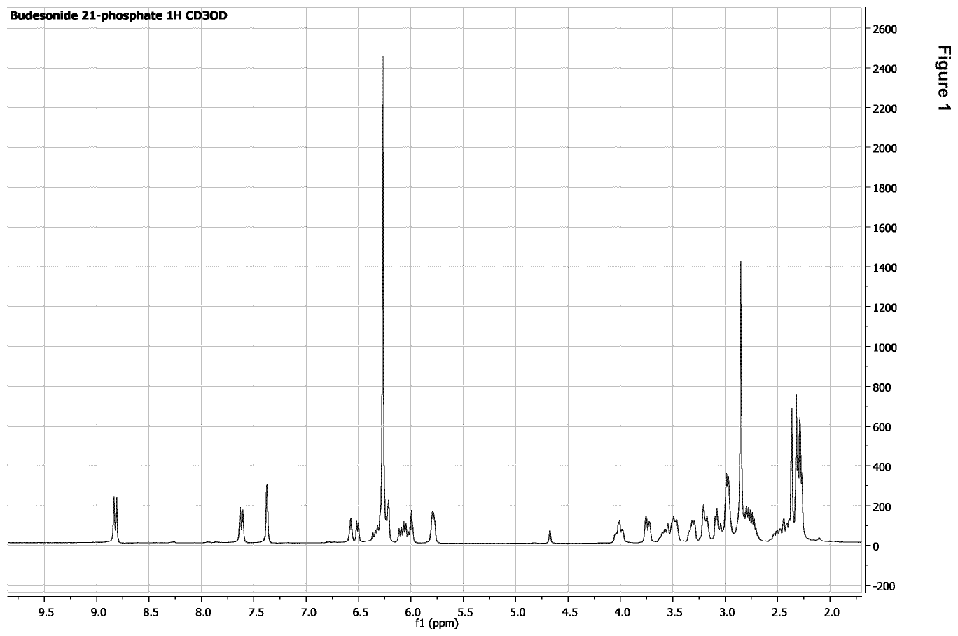

FIG. 2 shows 13C-NMR (126 MHZ; CD₃OD-d₄) spectrum of Budesonide 21-phosphate.

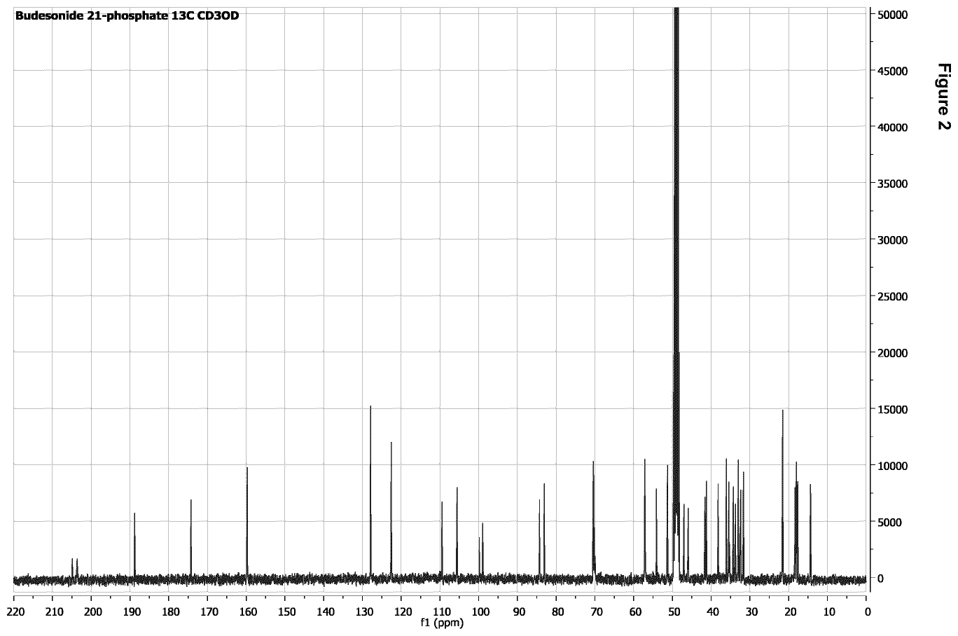

FIG. 3 shows 1H-NMR (500 MHZ; CD₃OD-d₄) spectrum of Budesonide 21-phosphate disodium salt.

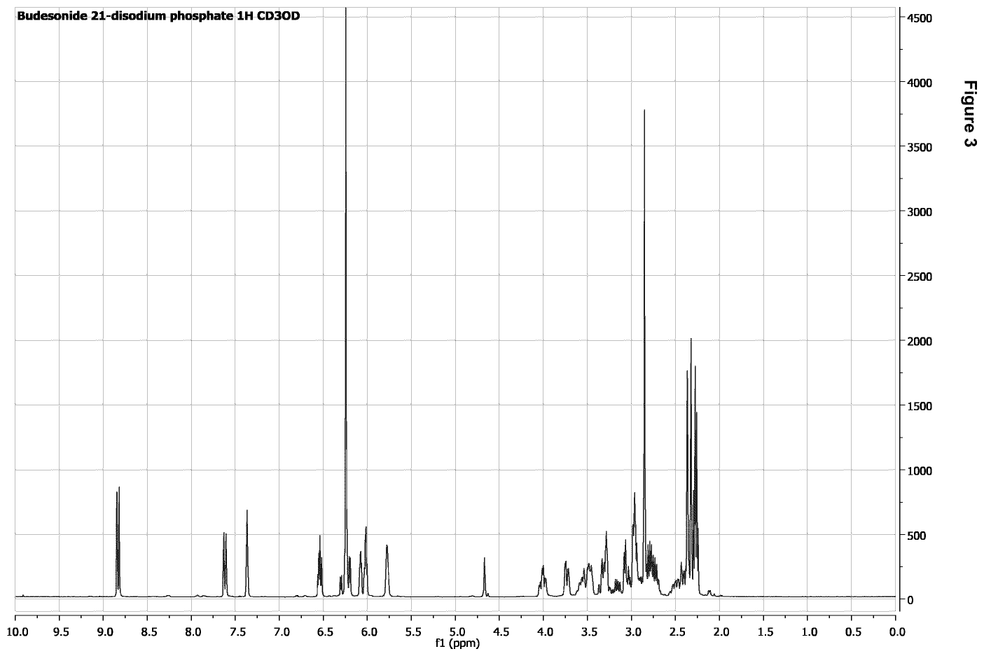

FIG. 4 shows 13C-NMR (126 MHZ; CD₃OD-d₄) spectrum of Budesonide 21-phosphate disodium salt.

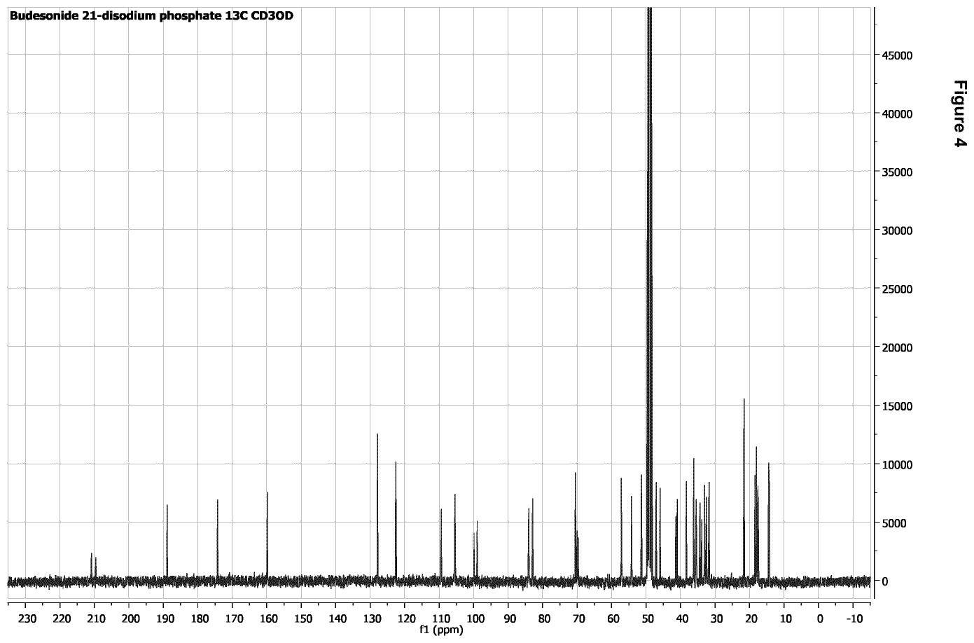

FIG. 5 shows ESI-MS spectrum of Budesonide 21-phosphate.

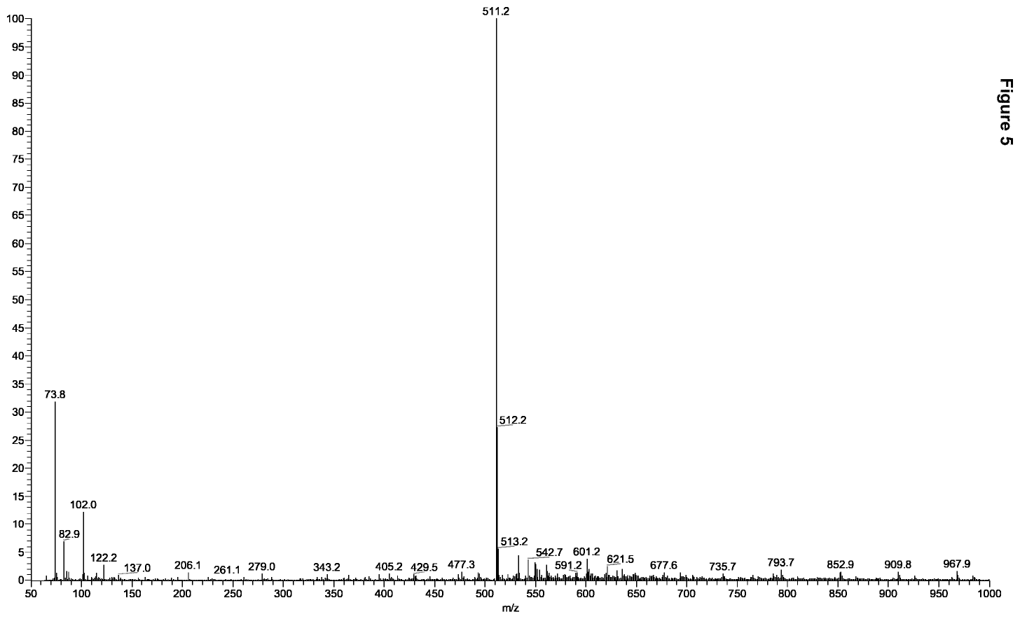

FIG. 6 shows the FT-IR spectrum of Budesonide 21-phosphate.

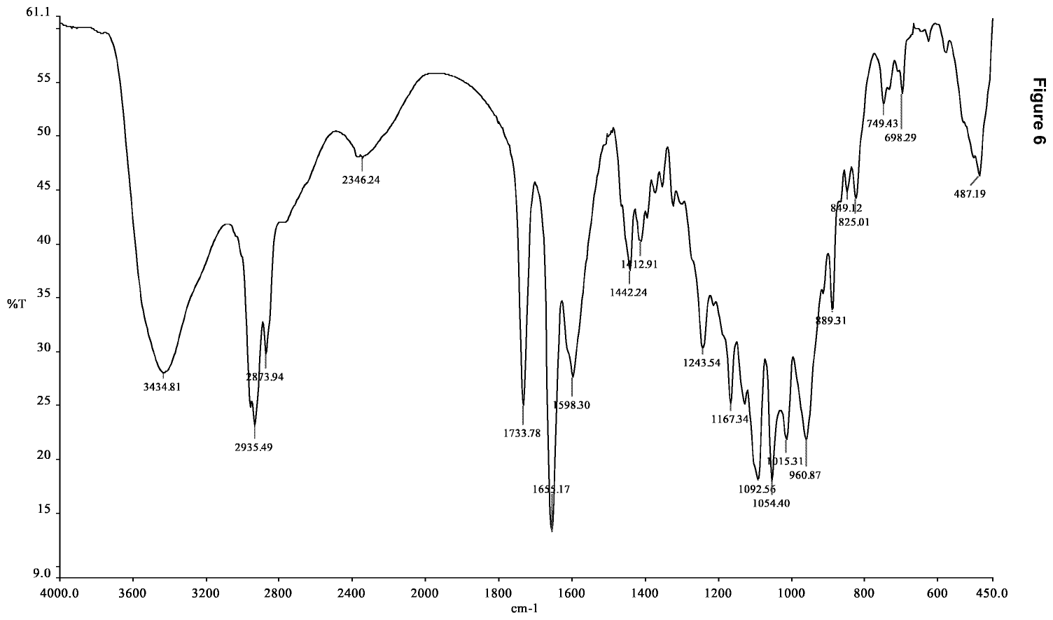

FIG. 7 shows the FT-IR spectrum of Budesonide 21-phosphate disodium salt.

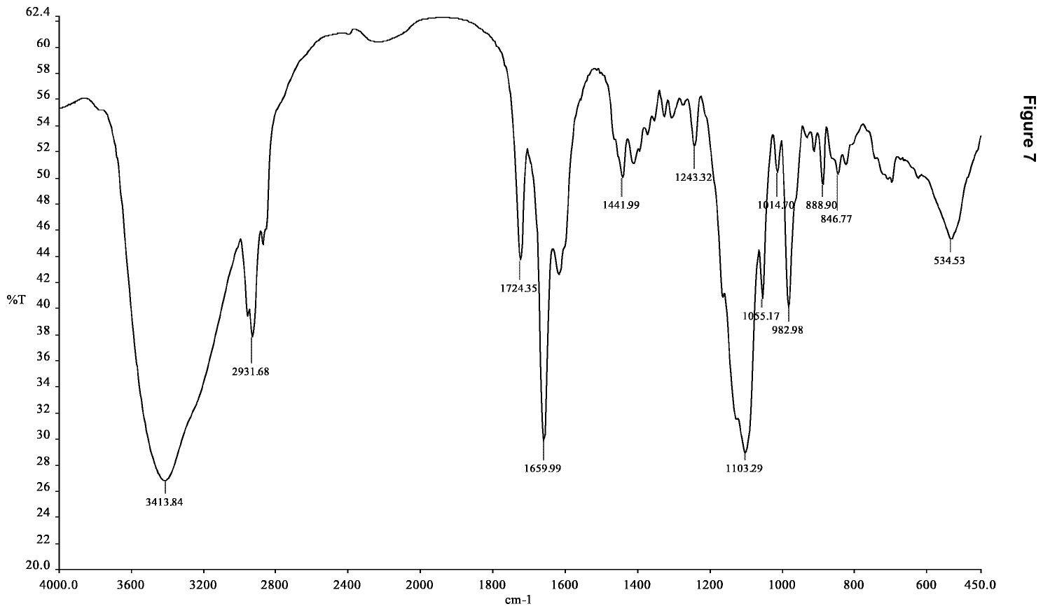

FIG. 8 shows the X-Ray Powder Diffraction spectrum of Budesonide 21-phosphate.

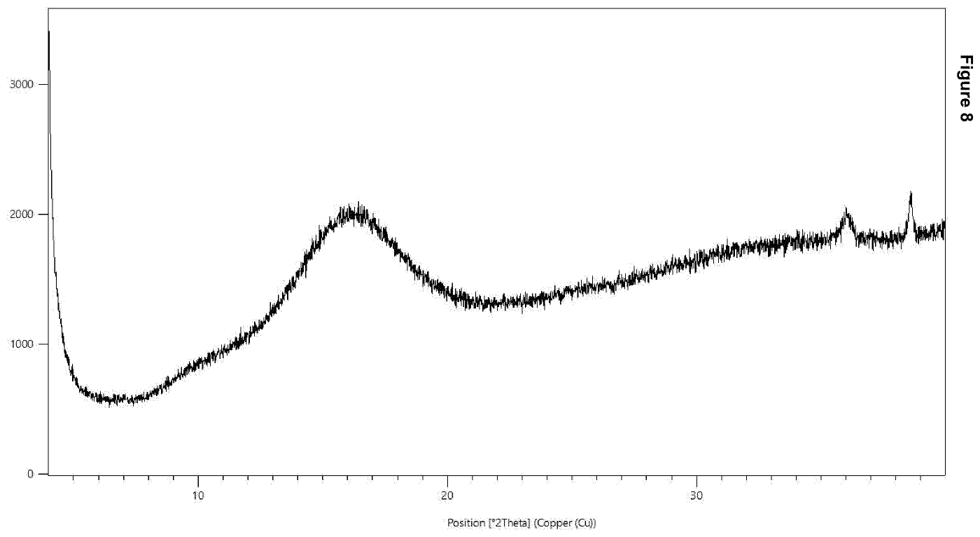

FIG. 9 shows the X-Ray Powder Diffraction spectrum of Budesonide 21-phosphate disodium salt.

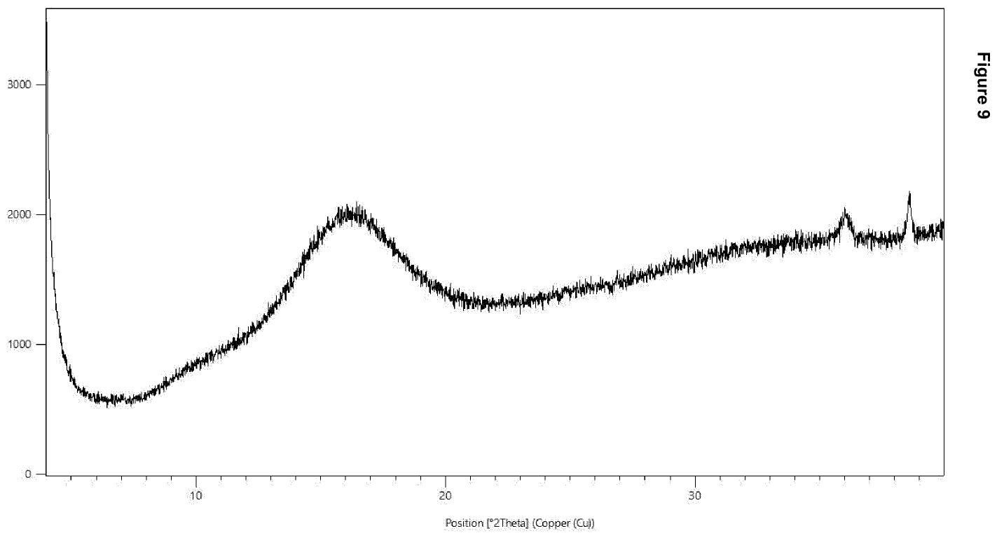

FIG. 10 shows UV spectrum of budesonide 21 phosphate disodium salt.

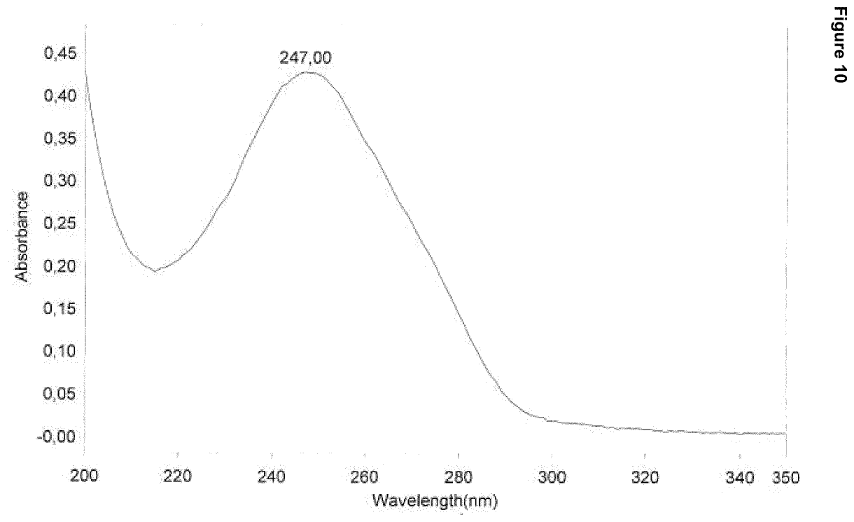

### DETAILED DESCRIPTION OF THE INVENTION

According to a first aspect, the present invention relates to a new process for preparing budesonide 21-phosphate of formula (II)

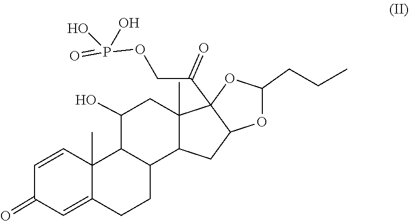 [SMILES: CCCC1OC2CC3C4CCC5=CC(=O)C=CC5(C)C4C(O)CC3(C)C2(C(=O)COP(=O)(O)O)O1]

which comprises the steps of:
a) reacting budesonide with tetrabutylammonium dihydrogen phosphate and trichloroacetonitrile to obtain the compound of formula (II);
b) optionally, satisfying the compound of formula (II) with NaOH to form the corresponding disodium salt.

Advantageously, the one-pot procedure by tetrabutylammonium dihydrogen phosphate and trichloroacetonitrile provides the budesonide 21-phosphate in improved yield (83%).

In one preferred embodiment, the step a) is performed in an aprotic solvent, preferably selected from acetonitrile, acetone, ethyl acetate, dichloromethane, or chloroform. More preferably, acetonitrile.

In another embodiment, the step a) is performed at room temperature.

According to a preferred embodiment of the process of the invention, budesonide 21-phosphate is isolated by crystallization. The useful solvents for said crystallization are ethyl acetate, n-hexane. More preferably, ethyl acetate.

In one preferred embodiment, the pH of step b) is from 7 to 9.

In another embodiment, the process further comprises the step of isolating the disodium salt.

Budesonide 21-phosphate disodium salt has a much higher water solubility than Budesonide and Budesonide 21-phosphate. Its solubility can be defined “freely soluble in water (100-1000 mg/mL)” and is equal to 110 mg/ml.

At the concentration of use (0.25 mg ml-4.0 mg ml), it is rapidly soluble and remains stable at room temperature for long periods of time (12 months) without yellowing or precipitating.

According to a preferred embodiment of the process of the invention, the isolation step of disodium salt is carried out by adding anti-solvent selected from methanol or ethanol.

According to a preferred embodiment of the process of the invention, the isolation step of the disodium salt is carried out by treatment with an opportune solvent. The useful solvents for said process are diethyl ether, ethyl acetate, or n-hexane. More preferably, diethyl ether.

According to a second aspect, the present invention relates to budesonide 21-phosphate disodium salt having an amount of any single unknown impurity equal to or lower than 0.10% (by area %) or having an amount of the qualified impurities budesonide (I) or budesonide 21-phosphate (II) equal to or lower than 0.2% (by area %).

According to a third aspect thereof, the present invention relates to budesonide 21-phosphate disodium salt having a purity equal to or greater than 98% by area %.

Purity was assessed through the HPLC method of the European Pharmacopoeia 1075-Budesonide-related substance.

Budesonide 21-phosphate disodium salt contains only the process impurities, namely budesonide (RRT of 17.8 min) and budesonide 21-phosphate (RRT of 4.3 min), the RRT being measured using the same HPLC method of the European Pharmacopoeia.

### Chemistry

### Materials and Methods

All the commercial products have been purchased from Merck-Sigma Aldrich. ¹H (500 MHZ) and ¹³C (125 MHZ) NMR spectra were recorded on an Agilent INOVA spectrometer; chemical shifts were referenced to the residual solvent signal (CD₃OD: δ_{H}=3.31, δ_{C}=49.0). ESI-MS spectrum was recorded on a LTQ Orbitrap XL™ Fourier transform mass spectrometer (FTMS) equipped with an ESI ION MAX™ (Thermo Fisher, San José, USA). X-ray powder diffraction (XRPD) was performed using a Panalytical X'pert PRO diffractometer. Intensity profiles were collected in the 2θ range of 4-40° using Ni-filtered CuKα radiation (λ=1.5406 Å) at 40 kV and 30 mA, with a step size 0.02°, at a scanning time of 120 s/step. The diffraction patterns were processed using the Highscore Plus suite. IR spectra were recorded on Thermo Nicolet 5700 FT-IR spectrometer. Thermo Fisher GENESYS™ 40/50 Vis/UV-Vis Spectrophotometers.

### Example 1

### Preparation of Budesonide 21-Phosphate

To a solution of budesonide (200 mg, 0.46 mmol) in acetonitrile (1 mL), trichloroacetonitrile (220 mL, 2.20 mmol) is added, followed by dropwise addition of tetrabutylammonium dihydrogen phosphate (625 mg, 1.84 mmol) in acetonitrile (2 mL). The reaction mixture was monitored by TLC using CHCl₃/MeOH/CH₃COOH (8 mL/2 mL/150 mL) as eluent mixture. The reaction mixture is stirred at room temperature for 24 hours. The reaction was treated with 1 N NaOH and extracted with ethyl acetate. The aqueous phase was made acidic using a 1 N HCl solution and extracted several times with ethyl acetate. The combined organic phases were washed with brine, dried over sodium sulfate, and concentrated to give 195 mg of budesonide 21-phosphate (yield 83%). M.P. 219-221° C. LRMS (ES) (M+H)⁺: calcd, 510.5; found, 511.2.

¹H NMR (500 MHZ, CD₃OD-d₄) δ 7.47 (d, J=10.1 Hz, 1H), 6.26 (d, J=10.1 Hz, 1H), 6.01 (s, 1H), 5.18 (dd, J=13.1, 6.2 Hz, 1H), 4.96-4.83 (m, 2H), 4.72-4.62 (m, 2H), 4.41 (d, J=3.5 Hz, 1H), 2.64 (dt, J=13.0, 6.7 Hz, 1H), 2.37 (d, J=11.0 Hz, 1H), 2.24-2.09 (m, 3H), 1.94 (dd, J=17.8, 9.7 Hz, 1H), 1.70 (dd, J=14.1, 6.6 Hz, 1H), 1.60 (dd, J=12.1, 7.0 Hz, 3H), 1.51-1.39 (m, 4H), 1.04-0.89 (m, 7H).

¹³C NMR (126 MHZ, CD₃OD-d₄): δ 210.89, 209.55, 188.85, 174.28, 159.86, 127.84, 122.55, 109.41, 105.45, 99.88, 98.97, 84.01, 82.92, 70.53, 70.48, 69.96, 69.68, 57.17, 57.08, 54.22, 51.33, 47.07, 45.98, 45.94, 41.34, 40.97, 38.27, 36.17, 35.50, 35.35, 34.34, 33.83, 33.01, 32.47, 31.75, 21.55, 18.44, 17.98, 17.82, 17.54, 14.40, 14.26.

### Example 2

### Preparation of Budesonide 21-Phosphate Disodium Salt

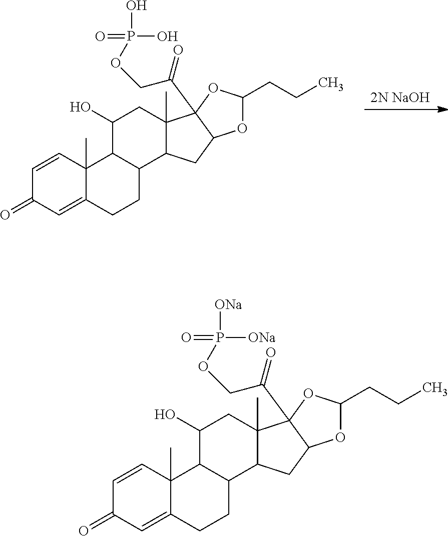

Budesonide 21-phosphate (100 mg, 0,196 mmol) was suspended in water (10 mL) and titrated with 2N NaOH to pH 7.94, obtaining a completely clear solution. Then the solvent was removed, and the residue was treated with methanol (5 mL) keeping the suspension at the boiling point of the solvent for 30 min. After cooling, the insoluble solid was filtered off and the solvent was removed in vacuo. The residue was then treated with diethyl ether affording budesonide 21-phosphate disodium salt as a white solid (86 mg, yield 79%), M.P. 245-246° C. 1H NMR (500 MHZ, CD₃OD-d₄) δ 7.47 (d, J=10.1 Hz, 1H), 6.26 (d, J=10.1 Hz, 1H), 6.01 (s, 1H), 5.18 (dd, J=13.1, 6.2 Hz, 1H), 4.96-4.83 (m, 2H), 4.72-4.62 (m, 2H), 4.41 (d, J=3.5 Hz, 1H), 2.64 (dt, J=13.0, 6.7 Hz, 1H), 2.37 (d, J=11.0 Hz, 1H), 2.24-2.09 (m, 3H), 1.94 (dd, J=17.8, 9.7 Hz, 1H), 1.70 (dd, J=14.1, 6.6 Hz, 1H), 1.60 (dd, J=12.1, 7.0 Hz, 3H), 1.51-1.39 (m, 4H), 1.04-0.89 (m, 7H).

¹³C NMR (126 MHZ, CD₃OD-d₄): δ 210.89, 209.55, 188.85, 174.28, 159.86, 127.84, 122.55, 109.41, 105.45, 99.88, 98.97, 84.01, 82.92, 70.53, 70.48, 69.96, 69.68, 57.17, 57.08, 54.22, 51.33, 47.07, 45.98, 45.94, 41.34, 40.97, 38.27, 36.17, 35.50, 35.35, 34.34, 33.83, 33.01, 32.47, 31.75, 21.55, 18.44, 17.98, 17.82, 17.54, 14.40, 14.26.

### Stability Testing

According to ICH guidelines (STABILITY TESTING OF NEW DRUG SUBSTANCES AND PRODUCTS Q1A (R2) Current Step 4 version dated 6 Feb. 2003), studies on budesonide 21-phosphate disodium salt were conducted at 25° C./60% RH (relative humidity) and 30° C./65% RH for a period of 12 months (m). Accelerated tests were also conducted under conditions of 40° C./75% RH for 6 months (m), as shown in the following Tables.

ICH study at 25° C. and 60% RH

| | Specification | Time 0 | Time 3 m | Time 6 m | Time 9 m | Time 12 m |
|---|---|---|---|---|---|---|
| Title | 95.0-105.0% | 98.22% | 98.10% | 98.08% | 97.85% | 97.45% |
| known | ≤0.2% | 0.14% | 0.14% | 0.14% | 0.15% | 0.15% |
| impurities | | | | | | |
| unknown | ≤0.1% | 0.05% | 0.05% | 0.05% | 0.08% | 0.08% |
| impurities | | | | | | |

ICH study at 30° C. and 65% RH

| | Specification | Time 0 | Time 3 m | Time 6 m | Time 9 m | Time 12 m |
|---|---|---|---|---|---|---|
| Title | 95.0-105.0% | 98.22% | 97.50% | 97.28% | 97.00% | 94.55% |
| known | ≤0.2% | 0.14% | 0.20% | 0.25% | 0.25% | 0.30% |
| impurities | | | | | | |
| unknown | ≤0.1% | 0.05% | 0.15% | 0.15% | 0.40% | 0.40% |
| impurities | | | | | | |

ICH study at 40° C. and 75% RH

| | Specification | | Time 0 | | Time 3 m | | Time 6 m | |
|---|---|---|---|---|---|---|---|---|
| Title | 95.0-105.0 | % | 98.22 | % | 96.50 | % | 93.28 | % |
| known | ≤0.2 | % | 0.14 | % | 0.30 | % | 0.45 | % |
| impurities | | | | | | | | |
| unknown | ≤0.1 | % | <0.05 | % | 2.45 | % | 2.80 | % |
| impurities | | | | | | | | |

### Formulation Studies

The following formulations were prepared to test the stability of the budesonide 21-phosphate disodium salt in aqueous solution.

Budesonide disodium phosfate

| | | | |
|---|---|---|---|
| Ingredient | Formula I | Formula II | Function |

Active substance

| | | | |
|---|---|---|---|
| Budesonide | 0.25 mg | 4.0 mg | Active substance |
| (disodium phosphate) | | | |

Excipients

| | | | |
|---|---|---|---|
| Disodium edetate | 0.10 mg | 0 | Chelating agent |
| Sodium chloride | 8.50 mg | 3.0 | Tonicity agent |
| Citric acid, anhydrous | 0.28 mg | 0 | Buffering agent |
| Sodium citrate | 0.50 mg | 10.0 | Buffering agent |
| tribasic dihydrate | | | |
| Water for injections | q. s. to 1.00 ml | q. s. to 1.00 ml | Solvent |
| HCl | q. s. to pH 4.5 | q. s. to pH 7.75 | |

The same formulations were tested according to ICH guidelines in the stability studies at 25° C. and 40° C. and the results are reported below.

ICH study at 25° C. and 60% RH FORMULA I

| | Specification | Time 0 | Time 3 m | Time 6 m | Time 9 m | Time 12 m |
|---|---|---|---|---|---|---|
| Aspect | Clear solution | Compliant | Compliant | Compliant | Compliant | Compliant |
| | free from | | | | | |
| | visible | | | | | |
| | particles | | | | | |
| Title | 95.0-105.0% | 100.5% | 100.2% | 100.2% | 100.0 | 99.8% |
| known | ≤0.2% | 0.10% | 0.10% | 0.10% | 0.15% | 0.15% |
| impurities | | | | | | |
| unknown | ≤0.1% | 0.05% | 0.05% | 0.05% | 0.10% | 0.10% |
| impurities | | | | | | |
| pH | 4.0-5.0 | 4.5 | 4.5 | 4.5 | 4.5 | 4.5 |

ICH study at 40° C. and 75% RH FORMULA I

| | Specification | | | | | | | |
|---|---|---|---|---|---|---|---|---|
| | Clear solution | | | | | | | |
| | free from | | | | | | | |
| | visible | | Time 0 | | Time 3 m | | Time 6 m | |
| Aspect | particles | | Compliant | | Compliant | | Compliant | |
| Title | 95.0-105.0 | % | 100.5 | % | 100.2 | % | 100.2 | % |
| known | ≤0.2 | % | 0.10 | % | 0.15 | % | 0.20 | % |
| impurities | | | | | | | | |
| unknown | ≤0.1 | % | <0.05 | % | 0.1 | % | 0.1 | % |
| impurities | | | | | | | | |

| | | | | |
|---|---|---|---|---|
| pH | 4.0-5.0 | 4.5 | 4.5 | 4.5 |

ICH study at 25° C. and 60% RH FORMULA II

| | Specification | Time 0 | Time 3 m | Time 6 m | Time 9 m | Time 12 m |
|---|---|---|---|---|---|---|
| Aspect | Clear solution | Compliant | Compliant | Compliant | Compliant | Compliant |
| | free from | | | | | |
| | visible | | | | | |
| | particles | | | | | |
| Title | 95.0-105.0% | 101.2% | 101.2% | 101.0% | 100.6% | 99.8% |
| known | ≤0.2% | 0.10% | 0.10% | 0.10% | 0.10% | 0.20% |
| impurities | | | | | | |
| unknown | ≤0.1% | 0.05% | 0.05% | 0.10% | 0.10% | 0.10% |
| impurities | | | | | | |
| pH | 7.25-8.25 | 7.75 | 7.75 | 7.75 | 7.75 | 7.75 |

ICH study at 40° C. and 75% RH FORMULA II

| | Specification | | | | | | | |
|---|---|---|---|---|---|---|---|---|
| | Clear solution | | | | | | | |
| | free from | | | | | | | |
| | visible | | Time 0 | | Time 3 m | | Time 6 m | |
| Aspect | particles | | Compliant | | Compliant | | Compliant | |
| Title | 95.0-105.0 | % | 101.2 | % | 100.5 | % | 99.6 | % |
| known | ≤0.2 | % | 0.10 | % | 0.20 | % | 0.20 | % |
| impurities | | | | | | | | |
| unknown | ≤0.1 | % | 0.05 | % | 0.10 | % | 0.10 | % |
| impurities | | | | | | | | |

| | | | | |
|---|---|---|---|---|
| pH | 7.25-8.25 | 7.75 | 7.80 | 7.90 |

1-10. (canceled)

11. A process for preparing budesonide 21-phosphate of formula (II) 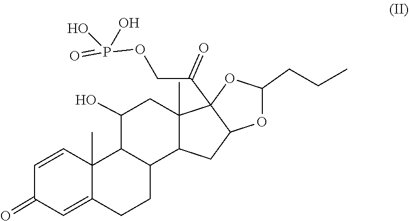 [SMILES: CCCC1OC2CC3C4CCC5=CC(=O)C=CC5(C)C4C(O)CC3(C)C2(C(=O)COP(=O)(O)O)O1]
comprising the steps of:
a) reacting budesonide with tetrabutylammonium dihydrogen phosphate and trichloroacetonitrile to obtain the compound of formula (II);
b) optionally, satisfying the compound of formula (II) with NaOH to form the corresponding disodium salt.

12. The process according to claim 11, wherein step a) is performed in an aprotic solvent.

13. The process according to claim 12, wherein the aprotic solvent is selected from acetonitrile, acetone, ethyl acetate, dichloromethane, and chloroform.

14. The process according to claim 13, wherein the aprotic solvent is acetonitrile.

15. The process according to claim 11, wherein step a) is performed at room temperature.

16. The process according to claim 11, wherein the pH of step b) is from 7 to 9.

17. The process according to claim 11, further comprising a step of isolating the compound of formula (II).

18. The process according to claim 17, wherein the compound of formula (II) is isolated by crystallization.

19. The process according to claim 18, wherein the compound of formula (II) is isolated by crystallization from ethyl acetate.

20. The process according to claim 11, further comprising a step of isolating the disodium salt of the compound of formula (II).

21. The process according to claim 20, wherein the isolation step of the disodium salt of the compound of formula (II) is carried out by adding an anti-solvent selected from methanol and ethanol.

22. The process according to claim 20, wherein the isolation step of the disodium salt of the compound of formula (II) is carried out by treatment with an opportune solvent.

23. The process according to claim 22, wherein the opportune solvent is selected from diethyl ether, ethyl acetate, and n-hexane.

24. The process according to claim 23, wherein the opportune solvent is diethyl ether.

25. Budesonide 21-phosphate disodium salt having an amount of any single unknown impurity equal to or lower than 0.10% by area % or having an amount of the qualified impurities budesonide (I) or budesonide 21-phosphate (II) equal to or lower than 0.2% by area %.

26. Budesonide 21-phosphate disodium salt having a purity equal to or greater than 98% by area %.
